(19) United States
(12) Patent Application Publication
(10) Pub. No.: US 2024/0318224 A1
(43) Pub. Date: Sep. 26, 2024

(54) METHOD OF TREATING DRUG RESISTANT ESKAPEE PATHOGENS USING THERAPEUTIC BACTERIOPHAGES

(71) Applicant: The Government of the United States, as Represented by the Secretary of the Army, FORT DETRICK, MD (US)

(72) Inventors: Andrey FILIPPOV, Germantown, MD (US); Mikeljon NIKOLICH, Tacoma Park, MD (US)

(21) Appl. No.: 18/579,769

(22) PCT Filed: Jul. 18, 2022
(86) PCT No.: PCT/US2022/073852

§ 371 Date: Jan. 16, 2024

Related U.S. Application Data
(60) Provisional application No. 63/222,551, filed on Jul. 16, 2021.

(51) Int. Cl. C12Q 1/18; C12N 7/00

(52) U.S. Cl. CPC C12Q 1/18; C12N 7/00; C12N 2795/00032

(57) ABSTRACT

Compositions and methods of improving an immunological response in a human subject suffering from a Pseudomonas aeruginosa in a wound comprising the step of:
(i) administering one or more isolated and purified strains of Pseudomonas aeruginosa and one or more pharmaceutically acceptable carriers or adjuvants to the wounded area of the human subject, said genomic phages interacting with innate and adaptive immune system of the human subject, while providing biological protection and/or wound recovery or stimulation; and
(ii) co-administering one or more additional genomic phages selected from genomic phages selected from Myoviridae, Podoviridae and Siphoviridae interacts and one or more pharmaceutically acceptable carriers or adjuvants to the wounded area of the human subject.

### STATEMENT AS TO RIGHTS OR INVENTIONS MADE UNDER FEDERALLY SPONSORED RESEARCH AND DEVELOPMENT

The invention was made with government support from the Bacterial Diseases Branch, Walter Reed Army Institute of Research (WRAIR). The United States Government has certain rights in the invention.

### SEQUENCE LISTING STATEMENT

The Sequence Listing, entitled ‘APT-28-PCT-WRAIR-21-06_SequenceListing_Jul. 18, 2022_Final.xml’ created on Jul. 18, 2022, and having a file size of 2,021,597 bytes is hereby incorporated by reference in its entirety.

### FIELD OF THE INVENTION

The invention relates to biologics to treat bacterial infections, such as multiple drug-resistant bacterial infections. The biologics comprise isolated and lytic bacteriophages and corresponding cocktails of isolated and lytic bacteriophages for treating ESKAPE pathogens.

### BACKGROUND

The quest to combat bacterial infections involving ESKAPE pathogens (Enterococcus spp., Staphylococcus aureus, Klebsiella pneumoniae, Acinetobacter baumannii, Pseudomonas aeruginosa, and Enterobacter spp.) impose therapeutic challenges due to the emergence of antimicrobial drug resistance. Recently, investigations with bacteriophages have led to the development of new approaches to treat ESKAPE infections. U.S. Pat. No. 10,676,721 discloses engineered bacteriophages expressing antimicrobial peptides or lytic enzymes or fragments thereof for targeting a broad spectrum of bacterial hosts, and for the long-term suppression of bacterial phage resistance for reducing bacterial infections. Bacteriophages have been demonstrated to be instrumental in the dissemination of virulence markers in ESKAPE pathogens. Significant challenges remain, however, for employing bacteriophage in treating ESKAPE pathogens and a knowledge gap exists in bacteriophage mediated antibiotic resistance and pathogenicity in ESKAPE infections. A bacteriophage infection can kill the host bacteria but in survivors can transfer genes which contribute towards survival of the pathogens in the host and resistance towards multiple antimicrobials. The knowledge on the dual role of bacteriophages in the treatment and pathogenicity will assist in the prediction and development of novel therapeutics targeting antimicrobial resistant ESKAPE pathogens. Inventors disclose herein the genome sequences of 10 Pseudomonas aeruginosa phages studied for their potential for formulation of a therapeutic cocktail; they represent the families Myoviridae, Podoviridae, and Siphoviridae. Genome sizes ranged from 43,299 to 88,728 nucleotides, with G_C contents of 52.1% to 62.2%. The genomes contained 68 to 168 coding sequences.

There still remains an unmet need to provide efficacious synthetic bacteriophages, bacteriophage cocktails and bacteriophages in combination with antibiotics are needed to develop effective therapeutics against ESKAPE infections.

### SUMMARY

This summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in any accompanying drawings and defined in the appended claims.

Specifically, a composition comprising (a) at least one phage selected from EPa8, EPa9, EPa15, EPa82, EPa83, or EPa87. In further embodiments, the composition comprises at least 2, 3, 4, 5, or 6 phages selected from EPa8, EPa9, EPa15, EPa82, EPa83, or EPa87.

In other preferred embodiments, the composition further comprises (a) at least one phage selected from EPa1, EPa2, EPa4, EPa5, EPa6, EPa7, EPa10, EPa11, EPa12, EPa13, EPa14, EPa16, EPa17, EPa18, EPa20, EPa21, EPa22, EPa24, EPa25, EPa26, EPa33, EPa38, EPa39, EPa40, EPa41, or EPa43.

In specific preferred embodiments, the composition comprises:
(a) EPa15 and any one of the phage selected from EPa1, EPa2, EPa4, EPa5, EPa6, EPa7, EPa10, EPa11, EPa12, EPa13, EPa14, EPa16, EPa17, EPa18, EPa20, EPa21, EPa22, EPa24, EPa25, EPa26, EPa33, EPa38, EPa39, EPa40, EPa41, or EPa43;
(b) EPa8 and any one of the phage selected from EPa1, EPa2, EPa4, EPa5, EPa6, EPa7, EPa10, EPa11, EPa12, EPa13, EPa14, EPa16, EPa17, EPa18, EPa20, EPa21, EPa22, EPa24, EPa25, EPa26, EPa33, EPa38, EPa39, EPa40, EPa41, or EPa43;
(c) EPa9 and any one of the phage selected from EPa1, EPa2, EPa4, EPa5, EPa6, EPa7, EPa10, EPa11, EPa12, EPa13, EPa14, EPa16, EPa17, EPa18, EPa20, EPa21, EPa22, EPa24, EPa25, EPa26, EPa33, EPa38, EPa39, EPa40, EPa41, or EPa43;
(d) EPa82 and any one of the phage selected from EPa1, EPa2, EPa4, EPa5, EPa6, EPa7, EPa10, EPa11, EPa12, EPa13, EPa14, EPa16, EPa17, EPa18, EPa20, EPa21, EPa22, EPa24, EPa25, EPa26, EPa33, EPa38, EPa39, EPa40, EPa41, or EPa43;
(e) Epa83 and any one of the phage selected from EPa1, EPa2, EPa4, EPa5, EPa6, EPa7, EPa10, EPa11, EPa12, EPa13, EPa14, EPa16, EPa17, EPa18, EPa20, EPa21, EPa22, EPa24, EPa25, EPa26, EPa33, EPa38, EPa39, EPa40, EPa41, or EPa43;
(f) EPa87 and any one of the phage selected from EPa1, EPa2, EPa4, EPa5, EPa6, EPa7, EPa10, EPa11, EPa12, EPa13, EPa14, EPa16, EPa17, EPa18, EPa20, EPa21, EPa22, EPa24, EPa25, EPa26, EPa33, EPa38, EPa39, EPa40, EPa41, or EPa43;
(g) EPa11 and EPa82;
(h) EPa11 and EPa83;
(i) EPa11 and EPa87;
(j) EPa82 and EPa83;
(k) EPa82 and EPa87;
(l) EPa83 and EPa87;
(m) EPa11, EPa39, EPa15, Epa83, and EPa87;
(n) EPa11, EPa39, EPa15, and Epa83;
(o) EPa11, EPa39, EPa15, and EPa87;
(p) EPa11, EPa15, EPa16, EPa18, EPa22, and EPa43;
(q) EPa11, EPa82, EPa83, and EPa87;
(r) EPa11, EPa39, EPa83 and EPa87; or
(s) EPa11, EPa15, EPa16, EPa18, EPa22 and EPa43.

In other preferred embodiments, the composition provides a dose of each phage in the range of 10⁵ to 10¹³ pfu, preferably, wherein said dose of each phage in the composition is in the range of 10⁶ to 10¹² pfu; 10⁷ to 10¹¹ pfu; 10⁸ to 10¹¹ pfu; 10⁹ to 10¹¹ pfu; 10⁹ to 10¹⁰ pfu; or even more preferably wherein said dose of each phage in the composition is approximately 10⁶ pfu, 10⁷ pfu, 10⁸ pfu, 10⁹ pfu, 10¹⁰ pfu, 10¹¹ pfu, 10¹² pfu, or 10¹³ pfu.

In preferred embodiments, composition causes lysis as measured by a change in:
(a) growth inhibition; (b) optical density; (c) metabolic output; (d) photometry (e.g., fluorescence, absorption, and transmission assays); and/or (e) plaque formation. Furthermore, phage-phage synergy, phage-antibiotic synergy and/or biofilm activity is capable of being measured in an lysis assay.

Additionally, in preferred embodiments, the change in photometry is measured using an additive that causes and/or enhances the photometric signal detection, preferably wherein said additive is tetrazolium dye.

In further embodiments, the composition is matched to the strains of ESKAPE bacterium known to be present in a geographic location.

In other preferred embodiments, the composition is formulated for IV injection, topical delivery, intraocular delivery, intranasal delivery, nebulization, intra-articular injection, oral delivery, intradermal delivery, or for IM injection.

As also described herein, a method of treating an infection caused by one or more bacterium, wherein said method comprises administering to a subject a therapeutically effective amount of the composition as described herein, wherein said composition is effective in treating and/or reducing said infection.

In preferred embodiments, the bacterium is multi-drug resistant; clinically refractory to antimicrobial treatment; clinically refractory to antimicrobial treatment due to biofilm production; and/or clinically refractory due to the subject's inability to tolerate antimicrobials due to adverse reactions.

In other preferred embodiments, the subject is suffering from a pathogen selected from: Pseudomonas aeruginosa; Enterococcus spp.; Staphylococcus aureus; Klebsiella pneumoniae; Acinetobacter baumannii; or Enterobacter spp.

In further preferred embodiments, the infection is selected from: a prosthetic joint infection (PJI), a chronic bacterial infection, an acute bacterial infection, a refractory infection, an infection associated with a biofilm, an infection associated with an implantable device, diabetic foot osteomyelitis (DFO), diabetic foot infection (DFI), a lung infection, such as those occurring in patients having cystic fibrosis (CF) or pneumonia, an urinary tract infection (UTI), a skin infection, such as acne or atopic dermatitis, such as conjunctivitis, bacterial kaeratitis, endophthalmitis, or blepharitis, sepsis or other blood infection.

In additional preferred embodiments, the device is permanently implanted in the subject; temporarily implanted in the subject; removable; and/or is selected from a prosthetic joint, a left-ventricular assist device (LVAD), a stent, a metal rod, an in-dwelling catheter, spinal hardware and/or instrumentation, and/or bone hardware and/or instrumentation.

As described herein the composition can be administered: by IV injection; by direct injection to the site of infection; topically; intraocularly; intranasally; by nebulization; by an intra-articular injection; by an IM injection; prophylactically; prior to a surgery; in lieu of surgery; during surgery; as a single occasion (i.e., as a “single shot”); and/or as a therapeutic course over 2, 3, 4, 5, 6, 7, 8, 9, 10, 11, 12, 13, or 14 weeks or more.

Additionally, in preferred embodiments, and as described herein is a method of treating drug resistant pathogens in a human subject presenting one or more wounds comprising the step of: administering a combination of genomic lytic bacteriophages to the human subject, wherein the drug resistant pathogens are selected from Enterococcus spp., Staphylococcus aureus, Klebsiella pneumoniae, Acinetobacter baumannii, Pseudomonas aeruginosa, and Enterobacter spp. and one or more pharmaceutically acceptable carriers or adjuvants. In a further embodiment, the one or more additional genomic phages are selected from Enterococcus spp., Staphylococcus aureus, Klebsiella pneumoniae, Acinetobacter baumannii, Pseudomonas aeruginosa, and Enterobacter spp.

A method of treating Pseudomonas aeruginosa in a human subject comprising the step of: administering a combination of genomic phages to the human subject, wherein the genomic phages represent families selected from Myoviridae, Podoviridae and Siphoviridae and one or more pharmaceutically acceptable carriers or adjuvants. In a further embodiment, the genomic phages are selected from Enterococcus spp., Staphylococcus aureus, Klebsiella pneumoniae, Acinetobacter baumannii, Pseudomonas aeruginosa, and Enterobacter spp.

A method of treating Pseudomonas aeruginosa in a human subject presenting a wound comprising the step of: administering a combination of genomic phages to the human subject, wherein the genomic phages represent families selected from Myoviridae, Podoviridae and Siphoviridae. In a further embodiment, the one or more additional genomic phages are selected from Enterococcus spp., Staphylococcus aureus, Klebsiella pneumoniae, Acinetobacter baumannii, Pseudomonas aeruginosa, and Enterobacter spp.

A method of treating wounds in a human subject suffering from a Pseudomonas aeruginosa comprising the step of: administering a combination of genomic phages to the human subject, wherein the genomic phages represent families selected from Myoviridae, Podoviridae and Siphoviridae. In a further embodiment, the one or more additional genomic phages are selected from Enterococcus spp., Staphylococcus aureus, Klebsiella pneumoniae, Acinetobacter baumannii, Pseudomonas aeruginosa, and Enterobacter spp.

A method of improving an immunological response in a human subject suffering from a Pseudomonas aeruginosa in a wound comprising the step of: administering a combination of genomic phages to the human subject, wherein the genomic phages represent families selected from Myoviridae, Podoviridae and Siphoviridae. In a further embodiment, the one or more additional genomic phages are selected from Enterococcus spp., Staphylococcus aureus, Klebsiella pneumoniae, Acinetobacter baumannii, Pseudomonas aeruginosa, and Enterobacter spp.

A method of improving an immunological response in a human subject suffering from a Pseudomonas aeruginosa in a wound comprising the step of: (i) administering one or more isolated and purified strains of Pseudomonas aeruginosa and one or more pharmaceutically acceptable carriers or adjuvants to the wounded area of the human subject, said genomic phages interacting with innate and adaptive immune system of the human subject, while providing biological protection and/or wound recovery or stimulation; and (ii) co-administering one or more additional genomic phages selected from genomic phages selected from Myoviridae, Podoviridae and Siphoviridae interacts and one or more pharmaceutically acceptable carriers or adjuvants to the wounded area of the human subject. In a further embodiment, the one or more additional genomic phages are selected from Enterococcus spp., Staphylococcus aureus, Klebsiella pneumoniae, Acinetobacter baumannii, Pseudomonas aeruginosa, and Enterobacter spp.

### BRIEF DESCRIPTION OF THE DRAWINGS

FIG. 1A shows the P. aeruginosa phage phylogenetic tree and FIG. 1B presents this same information in tabular format. Phages isolated in this work fell into the five highlighted clusters.

FIGS. 2A-2G shows the morphology of particles of P. aeruginosa bacteriophages isolated. Of the Myoviridae phages. FIG. 2A shows EPa3 (PB1-like);

FIG. 2B shows EPa7 (PB1-like); FIG. 2C shows EPa26 (PaP1-like). FIG. 2D shows a Podoviridae phage EPa4. FIG. 2E-G shows Siphoviridae phages. FIG. 2E shows EPa38 (M6-like); FIG. 2F shows EPa41 (Ab26-like); and FIG. 2G shows EPa33 (F116-like).

FIG. 3 shows the P. aeruginosa phages kill the host bacteria in biofilms (reduction by 2-7 logs.)

FIG. 4 shows the P. aeruginosa PAM2 phage cocktail efficacy in mice.

### DETAILED DESCRIPTION

The immune system has evolved to optimally respond to pathogens (see Janeway, C. A., Jr. Approaching the asymptote? Evolution and revolution in immunology. Cold Spring Harb Symp Quant Biol 54 Pt 1, 1-13, 1989; Zinkernagel, R. M., Science 271, 173-8, 1996; Germain, R. N., Nat Med 10, 1307-20, 2004). Immunization can be optimized, vaccine efficacy can be enhanced, by adopting characteristics of pathogens. For example, to enhance phagocytosis and antigen presentation, vaccines can be delivered in a particulate form with comparable dimensions to pathogens, such as emulsions, microparticles, iscoms, liposomes, virosomes and virus like particles to enhance phagocytosis and antigen presentation (O'Hagan, D. T. & Valiante, N. M. Nat Rev Drug Discov 2, 727-35, 2003). In addition, pathogen associated molecular patterns (PAMPs) stimulating the immune system as biological response modifiers, including toll-like receptors (TLR), can be used as adjuvants to activate antigen presenting cells and to enhance the immune response to vaccines (Johansen, P., et al., Clin Exp Allergy 35, 1591-1598, 2005b; O'Hagan, D. T. & Valiante, N. M. Nat Rev Drug Discov 2, 727-35, 2003; Krieg, A. M., Annu Rev Immunol 20, 709-60, 2002, each of which is incorporated herein by reference in its entirety). One key hallmark of pathogens is replication. Pathogen replication exposes the immune system to increasing amounts of antigen and immunostimulatory PAMPs over time.

In the context of limited success of antibiotics, phages are promising alternative antibacterials. Phages have demonstrated therapeutic efficacy against Pseudomonas aeruginosa infections in animals (1) and humans (1-3). Since P. aeruginosa phages have narrow host ranges (4, 5), phage cocktails are required to cover most clinical isolates (6). Inventors are developing a phage cocktail that is active against the majority of multi-drug resistant (MDR) P. aeruginosa isolates from traumatic and burn wounds. Here, we report the whole-genome sequences of 10 P. aeruginosa phages isolated from sewage (Table 1). Each phage lysed 23 to 58% of 156 diverse MDR isolates. The phages were complementary to each other (their mixes showed broader activity than single phages).

The phages were isolated from sewage collected in Washington, DC. P. aeruginosa strain PAO1 was used for enrichment. Phages were purified by three rounds of single-plaque isolation, propagated on strain PAO1 in broth, and concentrated by high-speed centrifugation as described previously (7). Host RNA and DNA were removed from lysates with RNase A and DNase, respectively, and phage DNA was isolated using proteinase K and SDS treatment followed by phenol-chloroform extraction, overnight precipitation with ethanol at 20° C., centrifugation, and resuspension in nuclease-free water (7). Phage DNA was sequenced using a Nextera XT DNA library preparation kit (Illumina, San Diego, CA). Libraries were validated and quantified using a TapeStation D5000 kit (Agilent Technologies, Inc., Santa Clara, CA) and an Invitrogen Qubit™ double-stranded DNA (dsDNA) broad-range (BR) assay kit (Thermo Fisher Scientific, Waltham, MA), respectively, purified with AMPure XP™ beads (Beckman CoulterDiagnostics, Brea, CA), and sequenced using a 600-cycle MiSeq reagent kit v3 on an Illumina MiSeq™ system, producing 300-bp paired-end reads. FastQC v0.11.5 (www.bioinformatics.babraham.ac.uk/projects/fastqc) was used for read quality control. Raw reads listed in Table 1 for each phage were subsequently trimmed using default parameters in Geneious Prime v2019.2.3 and were subjected to de novo assembly using default parameters in PATRIC (8). Phage genome annotations were carried out using the RAST server (9). Nucleic acid sequence similarity searches were performed using default parameters in BLASTn (10).

Phage genome sizes ranged from 43,299 to 88,728 nucleotides, with G_C contents of 52.1% to 62.2% (Table 1). The genomes contained 68 to 168 coding sequences. Phages EPa1 and EPa2 (family Podoviridae, genus Bruynoghevirus) were closely related to lytic phage LUZ24 (GenBank accession number AM910650.1) (11), based on BLASTn sequence comparisons. The phage genomes lacked significant nucleic acid sequence similarity to genes encoding integrases, recombinases, transposases, excisionases, and repressors of the lytic cycle. Therefore, EPa1 and EPa2 appear to be obligatorily lytic. Six Myoviridae phages, namely, EPa6, EPa11, EPa15, and EPa22 (genus Pbunavirus) and EPa17 and EPa24 (genus Nankokuvirus), also lacked genes typical of temperate phages, suggesting that they are strictly virulent, similar to other genus Pbunavirus (12) and Nankokuvirus (13) members. BLASTn and BLASTp analyses found no significant similarity in any of the eight phages to bacterial DNA and proteins, including drug resistance and pathogenicity determinants. Our data suggest that the eight phages are promising therapeutic candidates.

However, Siphoviridae phages EPa5 and EPa43, with high lytic potential, encoded putative proteins described as an integrase and a repressor in genome annotations of other phages, including Ab18, Ab19, Ab20, and Ab21, belonging to the genus Abidjanvirus (open reading frame 22 [ORF22] and ORF21 in the Ab18 genome [GenBank accession number LN610577]) (14). Subsequent inspection revealed only primase related domains and a lack of integrase-associated domains in the ORF22 product in EPa5, EPa43, and related phages. The ORF21 homolog contained an HTH_XRE domain, which is common in phages and has been associated with transcriptional antirepressor and repressor activities but remains largely uncharacterized. BLASTn and BLASTp searches for phages EPa5 and EPa43 did not identify any significant similarity to bacterial genes or proteins. Additional analysis is required to consider these two phages safe for therapeutic purposes.

In one embodiment, genomic phages useful for the invention were those selected and isolated from one or more P. aeruginosa strains. In particular, genomic phages useful for the invention were those selected and isolated from sewage collected in Washington, DC. A P. aeruginosa strain PAO1 was used for enrichment. The 10 complete phage genome sequences were deposited in GenBank and the NCBI Sequence Read Archive (SRA) under the accession numbers listed in Table 1. The composition may comprise one or more pharmaceutically acceptable carriers or adjuvants. It is contemplated that the methods of the invention can also be practiced with the addition of one or more pharmaceutically active carriers, which include adjuvants known in the art. An adjuvant is a substance or procedure which augments specific immune responses to antigens by modulating the activity of immune response in wounds, including wound cells. Exemplary adjuvants include salt based adjuvants such as alum salts, bacterial-derived adjuvants like lipopolysaccharides and bacterial toxins, adjuvant emulsions that enable the slow release of antigen, agonsitic antibodies to co-stimulatory molecules, Freunds adjuvant, muramyl dipeptides, and recombinant/synthetic adjuvants. In one embodiment, the adjuvant is a toll-like receptor (TLR) ligand, particularly a TLR-4, such as monophosphoryl lipid A (MPL), or TLR-7 ligand, such as R837. Alum is the most common salt-based adjuvant used to stimulate immune responses to protein vaccines and is the only adjuvant approved for human use in the United States (Alving, Vaccine 20(3):S56-S64 (2002); Hunter, Vaccine 20(3): 87-12 (2002)). However, alum favors Th2-biased responses and does not stimulate cell-mediated immunity. Mucosal immunity can be induced through the use of bacterial toxins such as cholera toxin (CT) and the E. coli heat labile enterotoxin (LT), however the safety' of these adjuvants is questionable (Alving, Vaccine 20(3):S56-S64 (2002); Hunter, Vaccine 20(3):S7-12 (2002)). The development of newer, safer adjuvants has recently focused on stimulating particular immune response pathways. Co-administration of cytokines, such as interferon-γ and granulocyte-macrophage colony stimulating factor (GM-CSF), has shown promise in stimulating cellular immune responses (reviewed in (Petrovsky and Aguilar, Immunol. Cell Biol. 82:488-496 (2004)). High levels of cytokines can cause toxicity however, and dosing regimens must be carefully modulated. Administration of cytokines has particular promise for DNA vaccination where genes encoding both the cytokine and antigen could be simultaneously expressed by the same vector. Additional adjuvants being explored include those that target the toll signaling pathway. CpG DMA motifs commonly found in bacterial DNA are potent activators of cellular immune responses, and newer generation DNA-based vaccines often encode multiple CpG motifs (reviewed in (Petrovsky and Aguilar, Immunol. Cell Biol. 82:488-496 (2004)).

Complete data on the P. aeruginosa phages disclosed herein are summarized in Table 2. Specifically, the GenBank and SRA Accession numbers (when available) are provided along with the SEQ ID NO: for each phage sequence. In preferred embodiments, the compositions and methods are performed with any combination of the phage, such as for example, at least one, at least two, at least three, at least four, at least five, at least six, at least seven, at least eight, at least nine, or at least ten phage selected from phage described in Table 2. The compositions can also include additional phage(s) not selected from the table below, so long as at least one of the phage listed in Table 1 are included in the composition.

TABLE 1
Genomic attributes of the 10 P. aeruginosa phages

| | | | Genome | G + C | No. of | Genome | No. of | GenBank | MTA |
|---|---|---|---|---|---|---|---|---|---|
| Phage | | | Length | Content | Protein- | Coverage | Raw | Accession | Accession |
| Name | Family | Genus | (bp) | (%) | Coding Genes | (x) | Reads | No. | No. |
| EPa1 | Podoviridae | Bruynoghevirus | 45,230 | 52.1 | 76 | 158.1 | 308,634 | MT108723 | SAMN15311669 |
| EPa2 | Podoviridae | Bruynoghevirus | 43,299 | 52.3 | 68 | 757.2 | 302,307 | MT108724 | SAMN15311670 |
| EPa5 | Siphoviridae | Abidjanvirus | 63,969 | 62.2 | 91 | 1672.2 | 534,271 | MT108725 | SAMN15311671 |
| EPa6 | Myoviridae | Pbunavirus | 66,031 | 55.1 | 95 | 70.4 | 202,626 | MT108726 | SAMN15311672 |
| EPa11 | Myoviridae | Pbunavirus | 66,800 | 55.7 | 95 | 1004.6 | 272,627 | MT108727 | SAMN15311673 |
| EPa15 | Myoviridae | Pbunavirus | 66,002 | 55.6 | 95 | 1197.8 | 479,511 | MT413450 | SAMN15311674 |
| EPa17 | Myoviridae | Nankokuvirus | 88,600 | 54.8 | 164 | 2099.6 | 671,404 | MT108728 | SAMN15311675 |
| EPa22 | Myoviridae | Pbunavirus | 65,897 | 55.4 | 98 | 1556.5 | 457,227 | MT108729 | SAMN15311676 |
| EPa24 | Myoviridae | Nankokuvirus | 88,728 | 54.8 | 168 | 4728.4 | 1,577,519 | MT108730 | SAMN15311677 |
| EPa43 | Siphoviridae | Abidjanvirus | 64,323 | 62 | 97 | 2003.0 | 398,955 | MT108731 | SAMN15311678 |

TABLE 2
Data on GenBank and SRA entries for P. aeruginosa phages

| Phage | SEQ ID | | | GenBank | Date of | SRA Accession | Date of |
|---|---|---|---|---|---|---|---|
| Name | NO: | Family | Genus | Accession No. | depositing | No. | depositing |
| EPa1 | 1 | Podoviridae | Bruynoghevirus | MT108723 | Apr. 1, 2020 | SAMN15311669 | Jun. 17, 2020 |
| EPa2 | 2 | Podoviridae | Bruynoghevirus | MT108724 | Apr. 1, 2020 | SAMN15311670 | Jun. 17, 2020 |
| EPa4 | 3 | Podoviridae | Bruynoghevirus | MT118288 | Apr. 1, 2020 | SRR13222827 | Dec. 9, 2020 |
| EPa5 | 4 | Siphoviridae | Abidjanvirus | MT108725 | Apr. 1, 2020 | SAMN15311671 | Jun. 17, 2020 |
| EPa6 | 5 | Myoviridae | Pbunavirus | MT108726 | May 10, 2020 | SAMN15311672 | Jun. 17, 2020 |
| EPa7 | 6 | Myoviridae | Pbunavirus | MT118289 | Apr. 1, 2020 | SRR13196079 | Dec. 3, 2020 |
| EPa8 | 7 | | | N/D | | | |
| EPa9 | 8 | | | N/D | | | |
| EPa10 | 9 | Myoviridae | Pbunavirus | MT118290 | Apr. 1, 2020 | SRR13196078 | Dec. 3, 2020 |
| EPa11 | 10 | Myoviridae | Pbunavirus | MT108727 | Apr. 1, 2020 | SAMN15311673 | Jun. 17, 2020 |
| EPa12 | 11 | Myoviridae | Pbunavirus | MT118291 | Apr. 1, 2020 | SRR13196070 | Dec. 3, 2020 |
| EPa13 | 12 | Myoviridae | Pbunavirus | MT118292 | Apr. 1, 2020 | SRR13196069 | Dec. 3, 2020 |
| EPa14 | 13 | Myoviridae | Pbunavirus | MT118293 | Apr. 1, 2020 | SRR13196068 | Dec. 3, 2020 |
| EPa15 | 14 | Myoviridae | Pbunavirus | MT413450 | Dec. 4, 2020 | SAMN15311674 | Jun. 17, 2020 |
| EPa16 | 15 | Myoviridae | Nankokuvirus | MT118294 | Apr. 1, 2020 | SRR13196067 | Dec. 3, 2020 |
| EPa17 | 16 | Myoviridae | Nankokuvirus | MT108728 | Apr. 1, 2020 | SAMN15311675 | Jun. 17, 2020 |
| EPa18 | 17 | Myoviridae | Nankokuvirus | MT118295 | Apr. 1, 2020 | SRR13196066 | Dec. 3, 2020 |
| EPa20 | 18 | Myoviridae | Pbunavirus | MT118297 | Apr. 1, 2020 | SRR13196064 | Dec. 3, 2020 |
| EPa21 | 19 | Myoviridae | Pbunavirus | MT118298 | Apr. 1, 2020 | SRR13196063 | Dec. 3, 2020 |
| EPa22 | 20 | Myoviridae | Pbunavirus | MT108729 | Apr. 1, 2020 | SAMN15311676 | Jun. 17, 2020 |
| EPa24 | 21 | Myoviridae | Nankokuvirus | MT108730 | Apr. 1, 2020 | SAMN15311677 | Jun. 17, 2020 |
| EPa25 | 22 | Myoviridae | Pbunavirus | MT118299 | Apr. 1, 2020 | SRR13196077 | Dec. 3, 2020 |
| EPa26 | 23 | Myoviridae | Nankokuvirus | MT118300 | Apr. 1, 2020 | SRR13196076 | Dec. 3, 2020 |
| EPa33 | 24 | Podoviridae | Hollowayvirus | MT118301 | Apr. 1, 2020 | SRR13196075 | Dec. 3, 2020 |
| EPa38 | 25 | Siphoviridae | Yuavirus | MT118302 | Apr. 1, 2020 | SRR13196074 | Dec. 3, 2020 |
| EPa39 | 26 | Myoviridae | Pbunavirus | MT118303 | Apr. 1, 2020 | SRR13196073 | Dec. 3, 2020 |
| EPa40 | 27 | Siphoviridae | Septimatrevirus | MT118304 | Apr. 1, 2020 | SRR13196072 | Dec. 3, 2020 |
| EPa41 | 28 | Siphoviridae | Septimatrevirus | MT118305 | Apr. 1, 2020 | SRR13196071 | Dec. 3, 2020 |
| EPa43 | 29 | Siphoviridae | Abidjanvirus | MT108731 | Apr. 1, 2020 | SAMN15311678 | Jun. 17, 2020 |
| EPa82 | 30 | | | N/D | | N/D | |
| EPa83 | 31 | | | N/D | | N/D | |
| EPa87 | 32 | | | N/D | | N/D | |

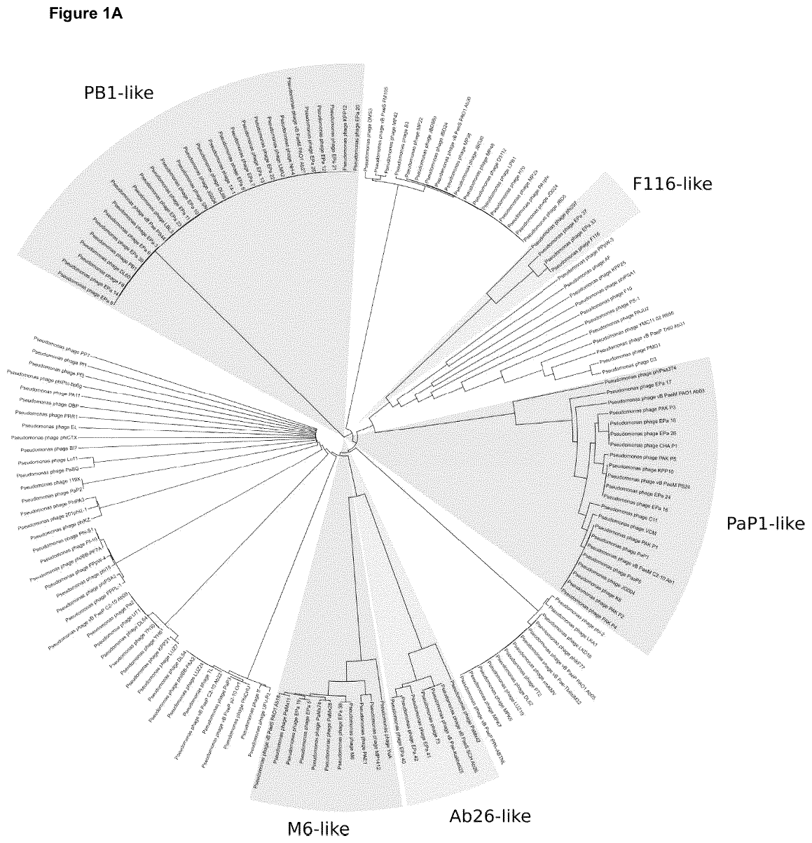

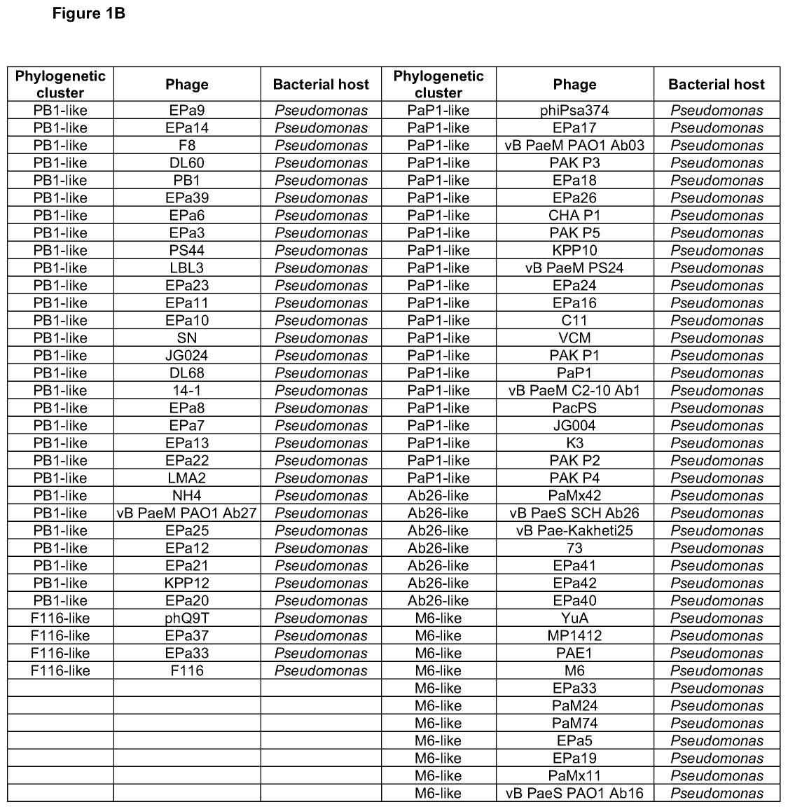

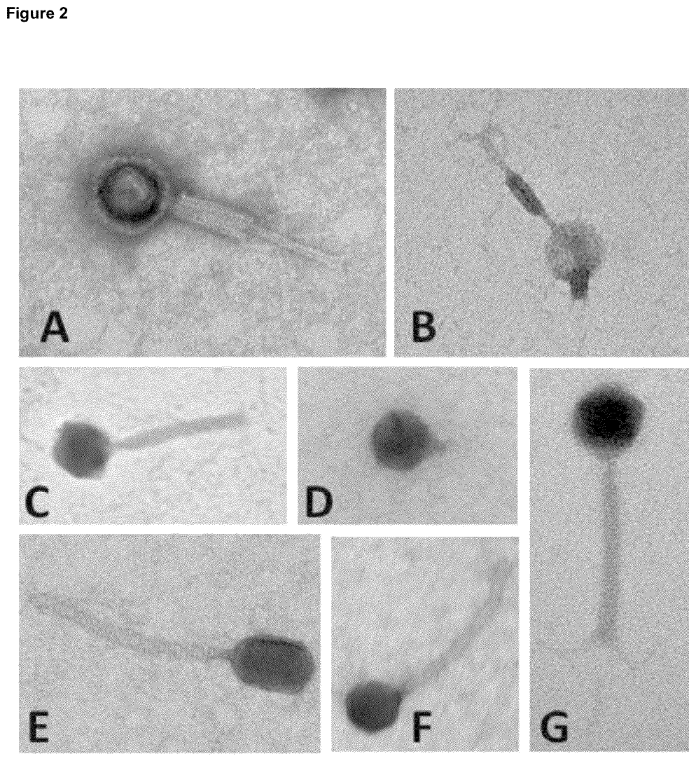

In one embodiment, one or more other genomic phages were used in combination with P. aeruginosa strains. For example, 30 new phages were isolated from sewage and environmental waters. Whole phage genomes were sequenced using an Illumina platform. The genome sizes varied from 32 to 89 kb, and the phages fell into five phylogenetic groups (FIG. 1): PB1-, F116-, PaP1-, Ab26-, and Mf-like. Morphology of phage particles studied using standard TEM (FIG. 2).

Thirty new different bacteriophages lytic for P. aeruginosa were isolated. Phages belong to five groups: PB1-, F116-, PaP1-. Ab26-, and M6-like, with genome sizes varying from 32 to 89 kb, within the families Myoviridae, Podoviridae and Siphoviridae. Phage lytic spectra tested on the panel of 54 strains including 51 MDR isolates from military hospitals ranged from 15% to 67%. Overall, 91% strains were susceptible to one or more phages from the panel. Seven phages demonstrated a marked lytic activity against P. aeruginosa PAO1 biofilms.

Inventors used rational design for developing P. aeruginosa durable fixed phage cocktails to include identification of phage receptors and testing their stability and activity in mixes. Five different receptors were identified for P. aeruginosa phages in different parts of LPS and type IV pilus. Five-phage cocktails PAM1 and PAM2 showed efficacy against lethal septicemic and local dorsal wound infections in mice. PAM2 is a stable defined mix of five phages with broad host range and high efficacy in preclinical studies and thus a promising therapeutic candidate.

In one embodiment, the rational design of the genomic phages is summarized in Table 3.

TABLE 3

| Steps | Methods | Quality Criteria |
|---|---|---|
| 1. Isolate phages | Single-plaque propagation (3×) | Plaque shape uniformity; plaque sizes may vary |
| 2. Propagate phages, isolate | Single-plaque propagation in 5 ml of broth; | Titers from n × 10¹⁰ to n × 10¹³ PFU/ml |
| genomic DNA | scaling up | |
| 3. Test lytic properties | Efficiency of plating; one-step growth; burst size | Clear plaques, robust lysis, limited to no secondary growth |
| 4. Characterize genomes | DNA Isolation, sequencing and analysis | Genomic purity; phage diversity; no detrimental genes |
| 5. Determine host ranges | Efficiency of plating on ≥100 strain diversity | 10-85% activity; determinable titer |
| | panel | |
| 6. Expand host ranges | Phage training; phage isolation on near- | Broader host ranges; phage activity against previously pan- |
| | neighbor species; phage engineering | phage-resistant bacteria |
| 7. Evaluate anti-biofilm activity | Live counts in biofilms; crystal violet OD | Killing effect; biofilm dispersal |
| | reduction; microscopy | |
| 8. Determine host resistance | Bacterial dilutions on phage in top agar (10⁹ | ≤10⁵ per cell per generation |
| rates | PFU/plate) | |
| 9. Identify host receptors | Mutagenesis of host genes; phage | Receptors should be different |
| | plating/adsorption | |
| 10. Assess immunogenicity | In vitro and in vivo cytokine assays; phage | Low immunogenicity and antigenicity; no/low pre- |
| | neutralization; pre-immunization impact on | Immunization impact on phage efficacy in vivo |
| | efficacy | |
| 11. Test stability | Storage at +4° C. and RT; repeated live counts | Stable at +4° C. for one year in SM buffer w/o gelatin |
| 12. Study phage interactions | Pairwise mixing; testing lytic activity/host | No impact on lysis/host range in mix vs. components; |
| | range/stability/resistance rates | expected reduction of phage resistance rates in mix |
| 13. Evaluate phage-antibiotic | Phage plaquing/lytic activity in presence of low | 100% or more increase in plaque sizes; 20% or more |
| interactions | antibiotic concentration; antibiotic MICs in the | acceleration of lysis; reduction of secondary bacterial |
| | presence of low-titer phage; combined phage- | growth; enhanced efficacy against biofilms and in animals |
| | antibiotic efficacy against biofilms and in vivo | |
| 14. Formulate cocktails | Mix 2-7 best synergistic or compatible phages | Good compatibility; 75-95% activity; anti-biofilm effect; host |
| | | resistance at ≤10⁶; stable at +4° C. for one year |
| 15. Purify phages for | CsCl gradient centrifugation, chromatography | ≤500 EU per dose for mice; ≤5 EU/kg/h for expanded access |
| animal/human use | | treatments |
| 16. Test experimentally in vivo | Wax worm model > mouse dorsal wound model > | Protection of wax worm from death; accelerated wound |
| | model target indication | healing in mice; bacterial burden reduction/wound |
| | | sterilization; protection from septicemia and death; clinical |
| | | recovery |
| 17. Use in expanded access | Testing MDR clinical isolates against phage | Pure, sterile, potent customized phage cocktail highly lytic |
| cases | panels, phage purification, sterilization, filling, | against the clinical isolate at 10⁹ PFU per dose in PBS with |
| | providing data to FDA and physician, phage | 10 mM of MgSO₄ |
| | shipping to the clinic | |
| 18. Manufacture a phage | Manufacture per cGMP requirements | Purity, sterility, reproducible potency, stability |
| cocktail in a GMP facility | | |
| 19. Test the product in Phase | Conduct human trials per FDA regulations | No adverse effects in Phase I study; significant efficacy in |
| I/II clinical trials | | Phase II study |

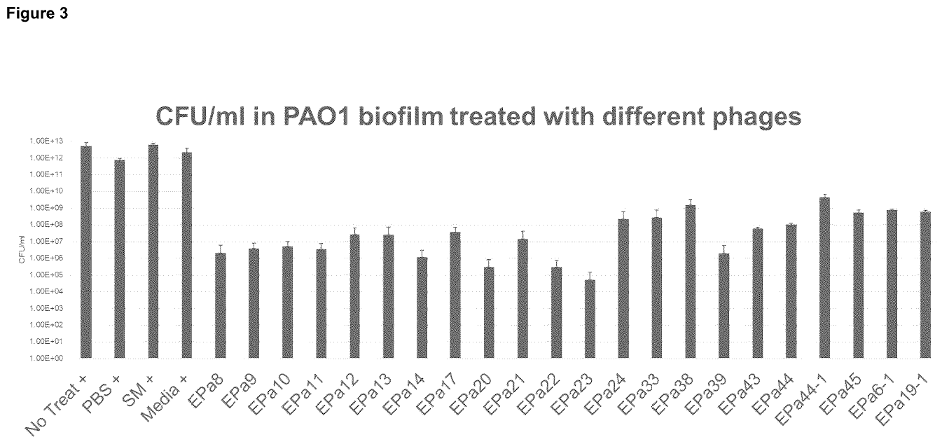

A panel of 83 lytic P. aeruginosa phages was isolated from sewage, environmental waters and soil. Whole genome sequencing and electron microscopy were used to classify these phages into three families and eight genera (FIG. 3), of which the representatives of five genera (marked in green) did not contain any bacterial DNA or putative determinants of transduction and thus can be safely used for human therapy.

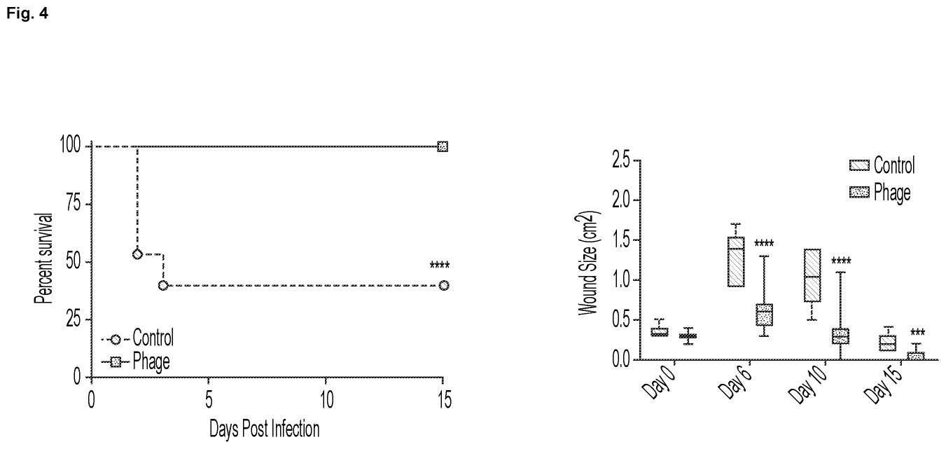

This phage panel was active against 165/186 (89%) of diverse clinical isolates from traumatic, burn and surgical wounds, abscesses, respiratory specimens, blood, and urine. Therapeutic phage cocktails were formulated based on phage high lytic activity, anti-biofilm effect (FIG. 4) broad host range, phylogenetic diversity, safe genome content, different host receptors, stability, and compatibility in mixes (Table 4).

TABLE 4
Therapeutic phage cocktails PAM2 and PAM3

| | | | | Host | | | | Interaction | |
|---|---|---|---|---|---|---|---|---|---|
| | | | | range | Host | | Resistance | with other | Mix |
| Mix | Phage | Family | Genus | (%) | receptor | Stability | rate | phages | activity |
| PAM2 | EPa11 | Myoviridae | Pbunavirus | 51 | L-OSA, | + | 7.54 × 10⁻⁶ | − | 76.4% |
| | | | | | VL-OSA | | | | |
| | EPa17 | Myoviridae | Nankokuvirus | 30 | LPS core | + | 2.19 × 10⁻⁷ | − | |
| | EPa22 | Myoviridae | Pbunavirus | 58 | L-OSA, | + | 3.42 × 10⁻⁶ | − | |
| | | | | | VL-OSA | | | | |
| | EPa24 | Myoviridae | Nankokuvirus | 47 | A, B | + | 9.39 × 10⁻⁶ | − | |
| | | | | | bands | | | | |
| | EPa43 | Siphoviridae | Abijanvirus | 33 | LPS core | + | <8.77 × 10⁻⁹ | − | |
| PAM3 | EPa11 | Myoviridae | Pbunavirus | 51 | L-OSA, | + | 7.54 × 10⁻⁶ | − | 81.2% |
| | | | | | VL-OSA | | | | |
| | EPa16 | Myoviridae | Nankokuvirus | 35 | L-OSA, | + | 1.33 × 10⁻⁶ | − | |
| | | | | | VL-OSA | | | | |
| | EPa17 | Myoviridae | Nankokuvirus | 30 | LPS core | + | 2.19 × 10⁻⁷ | − | |
| | EPa18 | Myoviridae | Nankokuvirus | 47 | A, B, Pil | + | ? | − | |
| | EPa22 | Myoviridae | Pbunavirus | 58 | L-OSA, | + | 3.42 × 10⁻⁶ | − | |
| | | | | | VL-OSA | | | | |
| | EPa39 | Myoviridae | Pbunavirus | 46 | L-OSA, | + | ? | − | |
| | | | | | VL-OSA | | | | |
| | EPa43 | Siphoviridae | Abijanvirus | 33 | LPS core | + | <8.77 × 10⁻⁹ | − | |

Plating phages on a mucoid pulmonary isolate of P. aeruginosa, we identified several host range mutants with broad activity against cystic fibrosis isolates (EPa82, EPa83, EPa87, see Table 5). A4-phagecocktail, PAM-CF1, consisting of one wild phage and three host range mutants was unexpectedly and surprisingly active against 40/47 (85%) of cystic fibrosis isolates.

TABLE 5
Phage activity against cystic fibrosis P. aeruginosa isolates.

| Phage | Family | Genus | Activity | Phage mix | Mix activity |
|---|---|---|---|---|---|
| EPa11 | Myoviridae | Pbunavirus | 19/47 (40%) | 11 + 22 | 19/47 (40%) |
| EPa22 | Myoviridae | Pbunavirus | 17/47 (36%) | 11 + 39 | 20/47 (43%) |
| EPa39 | Myoviridae | Pbunavirus | 18/47 (38%) | 82 + 83 | 29/47 (62%) |
| EPa82 | Podoviridae | Bruynoghevirus | 28/47 (60%) | 11 + 82 | 39/47 (83%) |
| EPa83 | Podoviridae | Bruynoghevirus | 28/47 (60%) | 11 + 82 + 83 + 87 (PAM-CF1) | 40/47 (85%) |

A rational design for developing P. aeruginosa durable fixed phage cocktails was employed in accordance with the invention, including anti-biofilm effects, identification of host receptors, testing phage stability and activity in mixes, and in vivo studies Six different receptors were identified for P. aeruginosa phages in different parts of LPS and type IV pilus. Five-phage cocktails PAM1 and PAM2 showed unexpected and surprisingly better efficacy against lethal septicemic and local dorsal wound infections in mice. Additionally, PAM2 and PAM3 components showed stability, full activity and resistance reduction when used as pairwise mixes. PAM3 is a defined and stable mix of six phages with unexpected and surprisingly broad host range (84%), suggesting a promising therapeutic candidate for human use.

### REFERENCES

1. Kortright K E, Chan B K, Koff J L, Turner P E. 2019. Phage therapy: a renewed approach to combat antibiotic-resistant bacteria. Cell Host Microbe. 252:219-232. doi.org/10.1016/j.chom.2019.01.014.
2. El Haddad L, Harb C P, Gebara M A, Stibich M A, Chemaly R F. 2019. A systematic and critical review of bacteriophage therapy against multidrug-resistant ESKAPE organisms in humans. Clin Infect Dis 69:167-178. doi.org/10.1093/cid/ciy947.
3. Jault P, Leclerc T, Jennes S, Pirnay J P, Que Y A, Resch G, Rousseau A F, Ravat F, Carsin H, Le Floch R, Schaal J V, Soler C, Fevre C, Arnaud I, Bretaudeau L, Gabard J. 2019. Efficacy and tolerability of a cocktail of bacteriophages to treat burn wounds infected by Pseudomonas aeruginosa (PhagoBurn): a randomised, controlled, double-blind phase 1/2 trial. Lancet Infect Dis 19:35-45. doi.org/10.1016/S1473-3099(18)30482-1.
4. Knezevic P, Kostanjsek R, Obreht D, Petrovic O. 2009. Isolation of Pseudomonas aeruginosa specific phages with broad activity spectra. Curr Microbiol 59:173-180. doi.org/10.1007/s00284-009-9417-8.
5. Kwiatek M. Mizak L, Parasion S, Gryko R, Olender A, Niemcewicz M. 2015. Characterization of five newly isolated bacteriophages active against Pseudomonas aeruginosa clinical strains. Folia Microbiol (Praha) 60:7-14. doi.org/10.1007/s12223-014-0333-3.
6. Merabishvili M, Pirnay J-P, De Vos D. 2018. Guidelines to compose an ideal bacteriophage cocktail. Methods Mol Biol 1693:99-110. doi.org/10.1007/978-1-4939-7395-8_9.
7. Sergueev K V, Filippov A A, Farlow J, Su W, Kvachadze L, Balarjishvili N, Kutateladze M, Nikolich M P. 2019. Correlation of host range expansion of therapeutic bacteriophage Sb-1 with allele state at a hypervariable repeat locus. Appl Environ Microbiol 85:e0109-19. doi.org/10.1128/AEM.01209-19.
8. Wattam A R. Davis J J, Assaf R, Boisvert S, Brettin T, Bun C, Conrad N, Dietrich E M, Disz T, Gabbard J L, Gerdes S, Henry C S, Kenyon R W, Machi D, Mao C, Nordberg E K, Olsen G J, Murphy-Olson D E, Olson R, Overbeek R, Parrello B, Pusch G D, Shukla M, Vonstein V, Warren A, Xia F, Yoo H, Stevens R L. Improvements to PATRIC, the all-bacterial Bioinformatics Database and Analysis Resource Center. Nucleic Acids Res. 2017 Jan. 4; 45(D1):D535-D542. doi: 10.1093/nar/gkw1017 www.ncbi.nlm.nih.gov/pmc/articles/PMC5210524/9.
9. Aziz R K, Bartels D, Best A A, et al. The RAST Server: rapid annotations using subsystems technology. BMC Genomics. 2008; 9:75. Published 2008 Feb. 8. Doi: 10.1186/1471-2164-9-75. www.ncbi.nlm.nih.gov/pmc/articles/PMC2265698/10.
10. Altschul S F, Gish W, Miller W, Myers E W, Lipman D J. 1990. Basic local alignment search tool. Journal of Molecular Biology 215(3):403-410 DOI 10.1016/S0022-2836(05)80360-2. www.sciencedirect.com/science/article/abs/pii/S0022283605803602?via %3Dihub
11. Ceyssens P-J, Hertveldt K, Ackermann H-W, Noben J-P, Demeke M, Volckaert G, Lavigne R. 2008. The intron-containing genome of the lytic Pseudomonas phage LUZ24 resembles the temperate phage PaP3. Virology 377:233-238. doi.org/10.1016/j.virol.2008.04.038.
12. Lavigne R, Darius P. Summer E J, Seto D, Mahadevan P, Nilsson A S, Ackermann H W, Kropinski A M. 2009. Classification of Myoviridae bacteriophages using protein sequence similarity. BMC Microbiol 9:224. doi.org/10.1186/1471-2180-9-224.
13. Morello E, Saussereau E, Maura D, Huerre M, Touqui L, Debarbieux L. 2011. Pulmonary bacteriophage therapy on Pseudomonas aeruginosa cystic fibrosis strains: first steps towards treatment and prevention. PLOS One 6(2):e16963. doi.org/10.1371/journal.pone. 0016963.
14. Essoh C, Latino L, Blouin Y, Loukou G, Nguetta S M, Lathro N S, Cablanmian A, Kra A, Midoux C, Vergnaud G, Pourcel C. 2015. Investigation of a large collection of Pseudomonas aeruginosa bacteriophages collected from a single environmental source in Abidjan, Cote d'lvoire. PLOS One 10(6):e0130548. doi.org/10.1371/journal.pone.0130548.

1-17. (canceled)

18. A composition comprising at least three phages selected from EPa8, EPa9, Epa15, EPa82, EPa83, and EPa87.

19. The composition of claim 18, wherein the composition causes lysis as measured by a change in Growth inhibition, Optical density, Metabolic output, Photometry, and/or Plaque formation, wherein the phage-phage synergy, phage-antibiotic synergy and/or biofilm activity is measured in a lysis assay.

20. The composition of claim 19, wherein the change in photometry is measured using an additive that causes and/or enhances the photometric signal detection, which additive is optionally tetrazolium dye.

21. A composition comprising the phages EPa83 and EPa87, and at least one phage selected from EPa1 EPa2, EPa4, EPa5, EPa6, EPa7, EPa10, EPa11, EPa12, EPa13, EPa14, EPa16, EPa17, EPa18, EPa20, EPa21, EPa22, EPa24, EPa25, EPa26, EPa33, EPa38, EPa39, EPa40, EPa41, and EPa43.

22. The composition of claim 21, wherein the composition consists of the phages EPa83, EPa87, EPa11, and EPa39.

22. The composition of claim 22, wherein the composition causes lysis as measured by a change in Growth inhibition, Optical density, Metabolic output, Photometry, and/or Plaque formation, wherein the phage-phage synergy, phage-antibiotic synergy and/or biofilm activity is measured in a lysis assay.

23. The composition of claim 22, wherein the change in photometry is measured using an additive that causes and/or enhances the photometric signal detection, which additive is optionally tetrazolium dye.

24. A composition comprising the phage EPa15 and at least one phage selected from EPa1 EPa2, EPa4, EPa5, EPa6, EPa7, EPa10, EPa11, EPa12, EPa13, EPa14, EPa16, EPa17, EPa18, EPa20, EPa21, EPa22, EPa24, EPa25, EPa26, EPa33, EPa38, EPa39, EPa40, EPa41, and EPa43.

25. The composition of claim 24, which consists of EPa15, EPa11, EPa16, EPa18, EPa22 and EPa43.

26. The composition of claim 24, wherein the composition causes lysis as measured by a change in Growth inhibition, Optical density, Metabolic output, Photometry, and/or Plaque formation, wherein phage-phage synergy, phage-antibiotic synergy and/or biofilm activity is measured in a lysis assay.

27. The composition of claim 26, wherein the change in photometry is measured using an additive that causes and/or enhances the photometric signal detection, which additive is optionally tetrazolium dye.
